(19) United States
(12) Patent Application Publication
(10) Pub. No.: US 2025/0204966 A1
(43) Pub. Date: Jun. 26, 2025

(54) ORTHOPEDIC CABLE BONE TRANSPORT DEVICE AND BONE TRANSPORT SYSTEM COMPRISING SAID DEVICE

(71) Applicants: Orthofix S.R.L., Bussolengo (IT); Texas Scottish Rite Hospital for Children, Dallas, TX (US)

(72) Inventors: Daniele Venturini, Povegliano Veronese (VR) (IT); Andrea Ottoboni, Giacciano con Baruchella (RO) (IT); Michael Lupatini, San Martino della Battaglia (BS) (IT); Davide Gaspari, Villafranca di Verona (VR) (IT); John D. Ross, Ovilla, TX (US); Mikhail L. Samchukov, Coppel, TX (US); Alexander M. Cherkashin, Flower Mound, TX (US); Karen D. Standefer, Flower Mound, TX (US)

(21) Appl. No.: 18/960,993

(22) Filed: Nov. 26, 2024

Related U.S. Application Data
(60) Provisional application No. 63/613,915, filed on Dec. 22, 2023.

(51) Int. Cl. A61B 17/84; A61B 17/88

(52) U.S. Cl. CPC A61B 17/842; A61B 17/8861

(57) ABSTRACT

The present invention relates to a medical cable (9) that may include a flexible main body (93) having an external cable diameter, the main body (93) having a first end (90) and a second end (91). The medical cable may also include at least one lead wire (98) extending from the first end (90) and/or from the second end (91) of the main body (93), the lead wire having a wire diameter and being more flexible than the main body (93), the wire diameter being less than one half of the cable diameter, said lead wire defining a closed loop (92).

### PRIORITY AND CROSS-REFERENCE TO RELATED APPLICATIONS

This application claims the benefit of the filing date of U.S. Application No. 63/613,915 filed Dec. 20, 2023, which is incorporated by reference herein in its entirety.

In addition, this application is related to U.S. application Ser. No. 17/560,789, filed Dec. 23, 2021, which is also incorporated by reference herein in its entirety.

### TECHNICAL FIELD

The present invention is generally directed to an orthopedic bone transport system and an associated medical cable.

The invention therefore has a useful application in the sector of orthopedics, in particular in bone defects treatment.

### BACKGROUND OF THE DISCLOSURE

External fixation systems are used in a variety of surgical procedures including fracture reduction, limb lengthening, and deformity correction, as well as treatment of non-unions, mal-unions, and bone defects. For bone defects treatment, a rigid framework comprising of upper (proximal) and lower (distal) external circular supports is placed externally around an affected limb and attached to associated upper (proximal) and lower (distal) bone segments using wires and/or pins. The proximal and distal external supports of the rigid framework are interconnected by threaded or telescopic rods stabilizing aligned spatial positioning of the distal bone segment relative to the proximal bone segment. One of those bone segments (e.g., proximal) is then divided into two bone segments (e.g., via osteotomy), thereby producing a third (intercalary) bone segment also called a transport bone segment that is gradually transported through the bone defect area creating newly formed bone tissues in the path of that transportation.

Stabilization of transport bone segment via cable, which significantly reduces soft tissue related complication with respect to alternative techniques, has been used for bone transport. However, traditional surgical methods using a transport cable, such as the Weber cable-bone transport or Balanced cable-bone transport, have the drawback of being extremely complicated. In particular, the practitioner has to dedicate a large amount of time to correctly insert the medical cable which is to be secured to the intercalary bone segment. Also, rollers and pulling rods occupy space on the external supports limiting places for other connecting and wire/pin fixation elements. Finally, the cable ends located between the proximal and distal external supports are unprotected and can be damaged from the outside failing to provide sufficient stability of transport bone segment fixation.

The technical problem underlying the present invention is therefore that of devising a cable bone transport system that solves at least some of the drawbacks of the prior art, and in particular a medical cable which can be advantageously employed in such a cable bone transport system.

### SUMMARY OF THE INVENTION

The idea for a solution forming basis of the present invention is that of modifying the traditional surgical technique and devising a medical cable with looped ends which can be easily routed through the bone segments during the surgical procedure. As described in the detailed description, the looped ends can be engaged by a hooking tool and then pulled within the bores drilled in the bone and within the intramedullary canal, thus greatly simplifying the task of the surgeon.

In view of the above, the technical problem is solved by a medical cable, comprising: a flexible main body having an external cable diameter, the main body having a first end and a second end; and at least one lead wire extending from the first end and/or from the second end of the main body, the lead wire having a wire diameter and being more flexible than the main body, the wire diameter being less than one half of the cable diameter, said lead wire defining a closed loop.

Preferably, two lead wires defining a closed loop are provided at both the first end and the second end.

Preferably, the first end of the cable is visibly distinguishable from the second end of the cable. This can be achieved through a different marking or a difference in color in the lead wires respectively attached to the first and second ends.

The wire diameter of the lead wire is preferably less than one third, even preferably less than one fifth of the cable diameter.

Preferably, the main body of the medical cable is formed of a plurality of strands, each strand having a plurality of strand wires.

Preferably, the strand wires and the lead wire are made of different materials; in particular the strand wire can be metal wires while the lead wire can be polymeric wires, for instance nylon wires.

Preferably, the lead wire forming the closed loop extends from the main body for at least 40 mm, ideally for at least 90 mm. Preferably, the lead wire is at most 200 mm, ideally at most 150 mm.

The length of the main body is preferably comprised between 500 mm and 1200 mm.

The medical wire according to the invention preferably comprises a dimensional index at the first end and/or at the second end.

The technical problem is also solved by an orthopedic bone transport system, comprising: an external fixation frame adapted to be solidly attached to at least a fixed bone segment; at least a cable pulling device-preferably two-adapted to be fixed on said external fixation frame; at least a medical cable adapted to secured to a transport bone segment and to be wound on said cable pulling device, said medical cable having closed loops at both its opposite ends.

The cable pulling device can further comprise a balance screw or pin adapted to be anchored to at least a fixed bone segment to redirect the medical cable within an intramedullary canal of said bone segment.

The technical problem is also solved by a drill guide for guiding the surgeon in implanting an orthopedic bone transport system, said drill guide comprising: a sleeve extending along a first axis having a first end portion configured to be received in an intramedullary canal of a bone; a first arm extending perpendicular to said sleeve along a second axis; a second arm attached to the first arm at a first joint; the second arm comprising a first guiding bore, the first guiding bore being spaced apart from the first joint along a direction parallel to the first axis; a third arm attached to the second arm at a second joint, the second joint being spaced apart from the first guiding bore along a direction parallel to a third axis, the third axis being orthogonal both to the first axis and to the second axis; the third arm comprising a second guiding bore, the second guiding bore being spaced apart from the second joint along a direction parallel to the second axis.

Preferably, the first guiding bore is parallel to the second axis and the second guiding bore is parallel to the third axis.

Preferably, the sleeve comprises a movable abutment which can be moved along the first axis to adjust the amount of length of the sleeve insertable in the intramedullary canal.

Preferably, the first joint and the second joint are rotatable so that the first arm, the second arm and the third arm can be set in a planar configuration for packaging, storage and/or shipping of the drill guide.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following. One or more features of any implementation or aspect may be combinable with one or more features of other implementation or aspect.

Features and advantages of the present invention will be disclosed with reference to the enclosed drawings relating to an indicative and a non-limiting implementation example.

### BRIEF DESCRIPTION OF THE DRAWINGS

The accompanying drawings illustrate implementations of the systems, devices, and methods disclosed herein and together with the description, serve to explain the principles of the present disclosure.

FIG. 1 shows a perspective view of a first embodiment of an orthopedic bone transport system according to the invention;

FIG. 2 shows a perspective view of a second embodiment of an orthopedic bone transport system according to the invention;

FIG. 3 shows a lateral view of a medical cable according to the invention;

FIG. 4A shows a median section of the medical cable of FIG. 1;

FIG. 4B shows an end section of the medical cable of FIG. 1;

FIG. 5A shows a lateral view of an end of a medical cable according to an alternative embodiment of the invention;

FIG. 5B shows a section taken along plane A-A of the medical cable of FIG. 5;

FIGS. 6-20 illustrate various stages of preparing a transport bone segment according to the method of implanting a medical cable according to the invention in an orthopedic bone transport system;

FIG. 21 shows a perspective view of a first embodiment of a cable balance screw being part of an orthopedic bone transport system according to the invention;

FIG. 22 shows a perspective view of a second embodiment of a cable balance screw being part of an orthopedic bone transport system according to the invention;

FIG. 23 shows a perspective view of a third embodiment of a cable balance screw being part of an orthopedic bone transport system according to the invention;

FIG. 24 shows a perspective view of a drill guide according to the invention;

FIG. 25 shows another perspective view of the drill guide of FIG. 24;

FIG. 26 shows a perspective view of the drill guide of FIG. 24 in a folded configuration;

FIG. 27 shows an exploded view of the drill guide of FIG. 24

These Figures will be better understood by reference to the following Detailed Description.

### DETAILED DESCRIPTION

In the following detailed description, reference is made to the accompanying drawings that form a part hereof and in which is shown, by way of illustration, specific embodiments. In the drawings, like numerals describe substantially similar components throughout the several views. Other embodiments may be disclosed, and structural changes may be made without departing from the scope of the present disclosure. The following detailed description is, therefore, not to be taken in a limiting sense.

The present disclosure refers, by way of example, to a segmental bone transport system 1 which adopts an innovative technique based on the pulling of a medical cable 9 by a cable pulling device 8 having a reel over which the medical cable 9 winds up. However, the medical cable according to the invention can also be employed with different segmental bone transport systems, such as a standard Balanced Cable bone transport system.

The bone transport system 1 is designed to apply a translation force to a transport bone segment 10 by decreasing the length of a flexible medical cable 9 that is attached to that transport bone segment 10. To do so, it adopts a concept which is similar to that used to tune stringed instruments. One end of the medical cable 9 is secured to the reel of the cable pulling device 8 onto which it is wound via a worm gear mechanism.

In the case of a musical string, the other end of the string is fixed and not allowed to translate. As the tuning mechanism is turned, the tension of the string is changed which also changes it resonate frequency. The worm gear mechanism ensures that the tension will be maintained when the mechanism is no longer being turned. The worm can turn the worm gear to tighten the cable, but the worm gear cannot turn the worm to release the tension on the cable.

In the bone transport system 1, tensioning the cable 9 by shortening it causes the bone transport segment 10 to move. The distance that the bone transport segment 10 can travel is a function of how much cable 9 can be wound around the reel of the cable pulling device 8.

Bone segment transport is performed in steps where the distance of transport for each step is preferably 0.25 to 0.50 mm performed two to four times a day but can be flexible depending on the bone formation. Also, it can be done by steps of 0.20 mm performed four or five times per day. In most cases, a total of 1.0 mm of transport distance per day is desired.

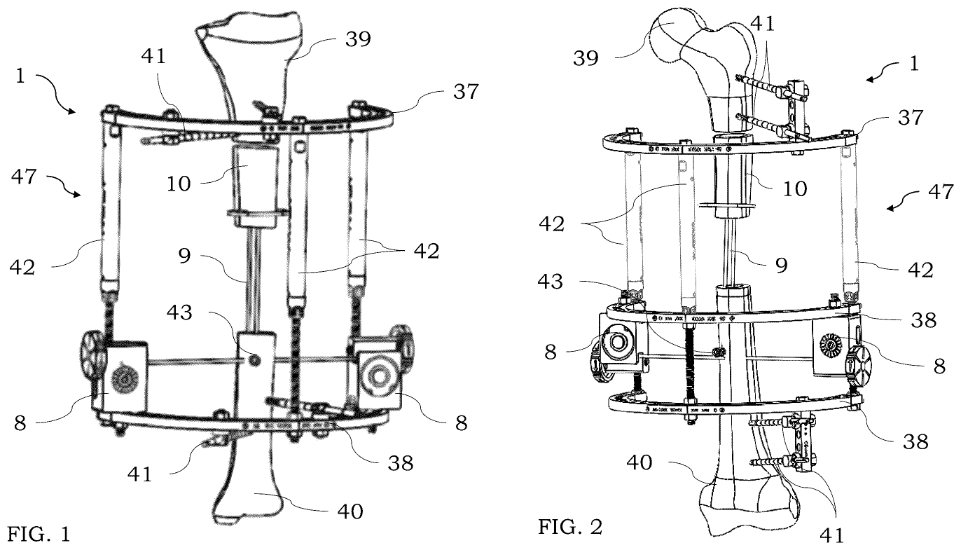

FIGS. 1 and 2 show examples of bone transport systems 1, 1′ which are provided with cable pulling devices 8 for bone transport and attached to bone segments according to embodiments of the present invention.

The bone transport system 1, 1′ according to the invention is meant to be applied to a long bone of a patient. In the embodiment of FIG. 1, the long bone is a tibia; in the embodiment of FIG. 2, the long bone is a femur.

The bone transport systems 1, 1′ comprise an external frame 47 which features a proximal ring 37 and at least one distal ring 38—two in the embodiment of FIG. 2. The proximal ring 37 and the distal ring 38 are respectively anchored to a proximal fixed bone segment 39 and to a distal fixed bone segment 40 by means of tensioned wires and/or fixation pins 41. The proximal ring 37 is connected to the distal ring 38 by means of longitudinal rods 42. Preferably, longitudinal rods 42 are extensible.

A transport bone segment 10 is axially comprised between the proximal fixed bone segment 39 and the distal fixed bone segment 40.

The bone transport system 1, 1′ comprises two cable pulling devices 8 which are attached via bolts at diametrically opposite sites on one of the distal rings 38. In the embodiment of FIG. 1, the cable pulling devices 8 are placed laterally and medially with respect to the distal fixed bone segment 40. In the embodiment of FIG. 2, the cable pulling devices 8 are placed anteriorly and posteriorly with respect to the distal fixed bone segment 40. In other embodiments, the cable pulling devices can be placed at different locations, and do not need to be diametrically opposite: for instance, one device can be placed anteriorly, and the other device can be placed medially. A medical cable 9 is pulled between the two cable pulling devices 1 and follows a path which will be described in the following.

The bone transport system 1 further comprises means 43 for redirecting the medical cable 9 at the distal fixed bone segment 40. Preferably, said means 43 for redirecting the medical cable 9 take the form of a fulcrum pin or screw which is implanted in the distal fixed bone segment 40, following an anterior-posterior direction.

Exemplary embodiments of a cable pulling device 1 that can be used with the bone transport systems 1, 1′ according to FIGS. 1 and 2 and may be found in U.S. application Ser. No. 17/560,789, filed Dec. 23, 2021, which is incorporated by reference in the present application.

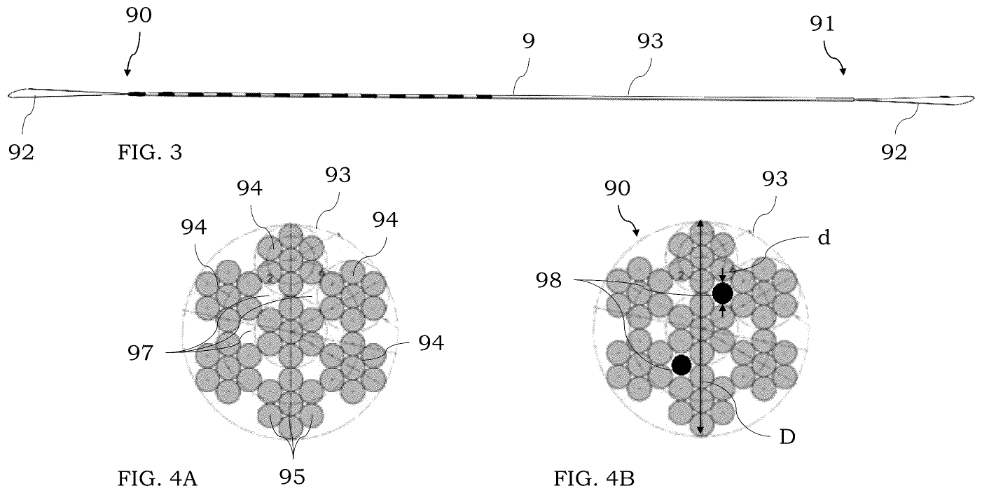

FIG. 3 shows an example of a medical cable 9 according to the invention. The medical cable 9 has a main body 93 having a first end 90 and a second end 91, opposite to the first end 90. The main body 93 preferably has an outer cable diameter D comprised between 1.6 mm and 2.2 mm and a total length comprised between 500 mm and 1200 mm.

At both the first end 90 and the second end 91 the medical cable 9 has closed loops 92. Said closed loops 92 are defined by lead wires 98 which are attached to the first end 90 and the second end 91 of the main body 93. The lead wire 98 has a wire diameter d which is much smaller than the cable diameter D, preferably less than one fifth of said cable diameter D. The lead wire 98, when extended from the respective end of the main body 93 to the tip of the closed loop 92, has a length comprised between 90 mm and 150 mm.

As best seen in FIG. 4A, the main body 93 of the medical cable 9 is formed by a plurality of strands 94, each made of a plurality of strand wires 95. The strand wires 95 are preferably metal wires, made for example of stainless steel or titanium. In the exemplary embodiment depicted herein, the main body 93 has seven strands 94: one central strand and six lateral strands disposed according to a honeycomb layout. Gaps 97 are defined at the intersection of the central strand with two adjacent lateral strength. Each single strand 94 has seven strand wires 95, also disposed with six lateral wires surrounding a central wire.

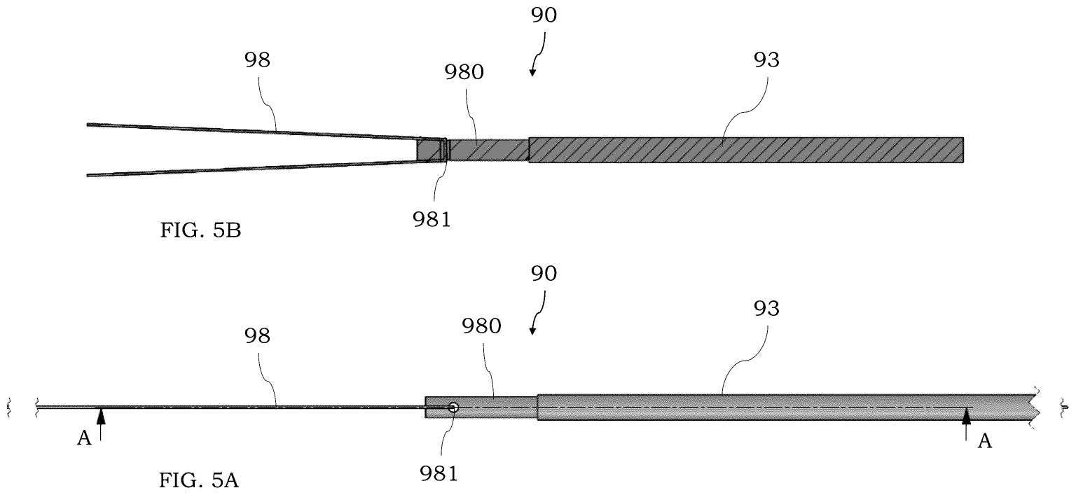

As best seen in FIG. 4B, the opposite ends of each lead wire 98 are inserted in two of the gaps 97, preferably two diametrically opposite gaps 97. The lead wire 98 is passed through the gap to secure it to the main body 93. In some examples, the lead wire 98 may be further secured to the main body 93 with an adhesive such as glue or tape. In some examples, a curable resin may be applied where the lead wire 98 passes through the main body 93 and a UV light or heat source may be used to cure the resin and affix the lead wire 98 to the main body 93. In some examples, a sleeve may be slid over the lead wire 98 and onto the main body 93. The sleeve may then be crimped in place to affix the lead wire 98 to the main body 93. In an example seen in FIGS. 5A, 5B a joint 980, for instance a stainless-steel joint, can be welded at one end either of the main body 93 or of one of its strands 94. The joint 980 features an eyelet 981 traversed by the lead wire 98. Various combinations of these techniques may be used to ensure the lead wire 98 remains secured to the main body 93.

In view of the above-described layout, the lead wire 98 has a wire diameter d which is smaller or equal to that of the gap 97, which is in turn comparable to the diameter of a single strand wire 95. The lead wire 98 can be made in a different material with respect to the strand wire 98, for instance a non-metallic material with a higher degree of flexibility such as a polymeric material, for instance nylon. Preferably, the opposite lead wires 98 have different colors, or else are provided with a visual or tactile marker to easily distinguish the first end 90 from the second end 91 of the medical cable 9. The main body 93 of the medical cable 9 can also have different colors or markings at its first end 90 and second ends 91.

The medical cable 9 enters the transport bone segment 10 at its resected end and is attached thereto by means of an external loop 44, passing through at least one first tangential bore 11 made on the transport bone segment 10 into its intermedullary canal and bone defect area following the bone axis, enters the fixed distal bone segment 40 from the intermedullary canal and, after orthogonal redirection around the fulcrum pin or screw 43, exits that distal bone segment 40.

The medical cable 9 is symmetrically stabilized on the transport bone segment 10 by making the two ends exit axially into the medullary canal.

In the distal fixed bone segment 40, the two ends of the medical cable 9 rotate around the fulcrum pin 43 and exit the cortices one on two different sides, through second and tangential bores 12.

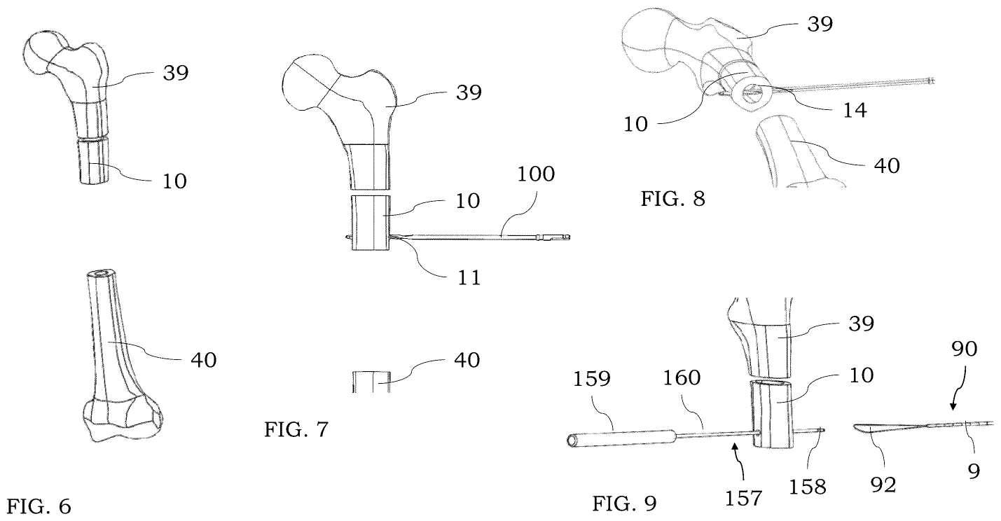

A bone transport system employing a medical cable 9 according to the invention can be implanted according to the advantageous surgical method described in the following with reference to FIGS. 6-21. The method is described herein with reference to a femur having a proximal fixed bone segment 39 and a distal fixed bone segment 40, but it can be equally applied to other long bones.

In a step of the surgical method depicted in FIG. 6, the proximal bone is cut to free the transport bone segment 10, in a per se known way.

In a step of the surgical method depicted in FIGS. 7 and 8, the first tangential bore 11 is drilled, by means of a drill tip 100, through the transport bone segment 10. In some examples, the first bore may extend in a medial-lateral direction although any suitable orientation may be used. It is observed that this step may be performed before the step of cutting the proximal bone so that, at this stage, the transport bone segment 10 may still be fixed to the proximal bone segment 39.

The first tangential bore 11 as a diameter which preferably at least twice the cable diameter D. As best seen in FIG. 8, the first tangential bore 11 is made at a short distance from the distal rim of the transport bone segment 10. Such a distance can be, for instance, of about 10 mm. The first tangential bore 11 traverses the intramedullary canal 14, preferably in its middle.

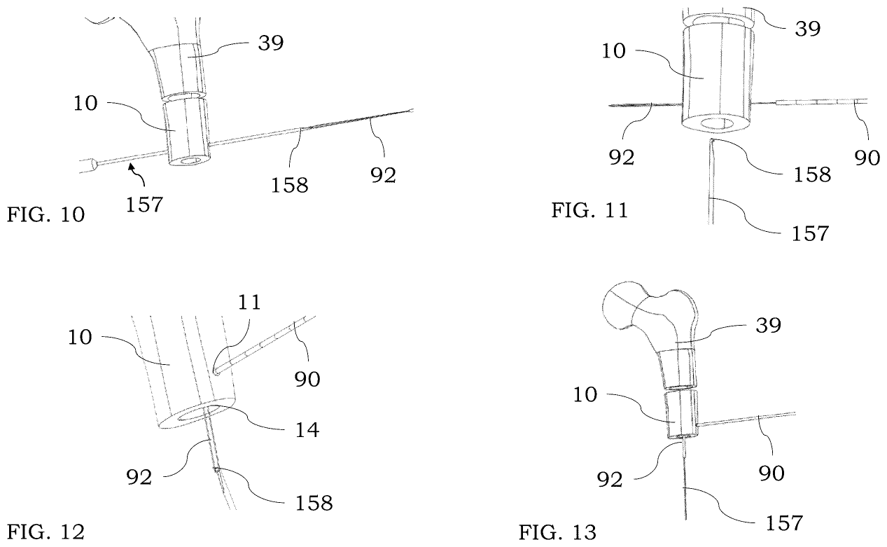

In a step of the surgical method depicted in FIGS. 9 and 10, a first end 90 of a medical cable 9 is inserted within the first tangential bore 11. As shown in the figures, a pulling instrument 157 may be used to facilitate routing of the medical cable 9 in the first tangential bore 11. The pulling instrument 157 comprises a hook 158, a handle 159, and a shaft 160 extending between the hook 158 and the handle 159. The hook 158 and the shaft 160 are passed through both cortices of the transport bone segment 10 in the first tangential bore 11. Then, the hook 158 engages the loop 92 defined by the lead wire 98 at the first end 90 of the medical cable 9. Finally, the hook 158 is retracted within the first tangential bore 11, pulling the cable's loop 92 through both cortices of the transport bone segment 10, so that it diametrically spans the intramedullary canal 14.

In a step of the surgical method depicted in FIGS. 11-13, the first end 90 of the medical cable 9 is routed through a distal opening of the intramedullary canal 14. First, as seen in FIG. 11, the pulling instrument 157 is inserted axially through the distal opening of the intramedullary canal 14. Then, the hook 158 engages the loop 92 spanning the intramedullary canal. After, as seen in FIGS. 12 and 13, the pulling instrument 157 is pulled distally to draw the loop 92 and the first end 90 of the medical cable 9 through the distal opening of the intramedullary canal 14. This technique greatly facilitates the bending of the thicker main body 93 of the medical cable 9.

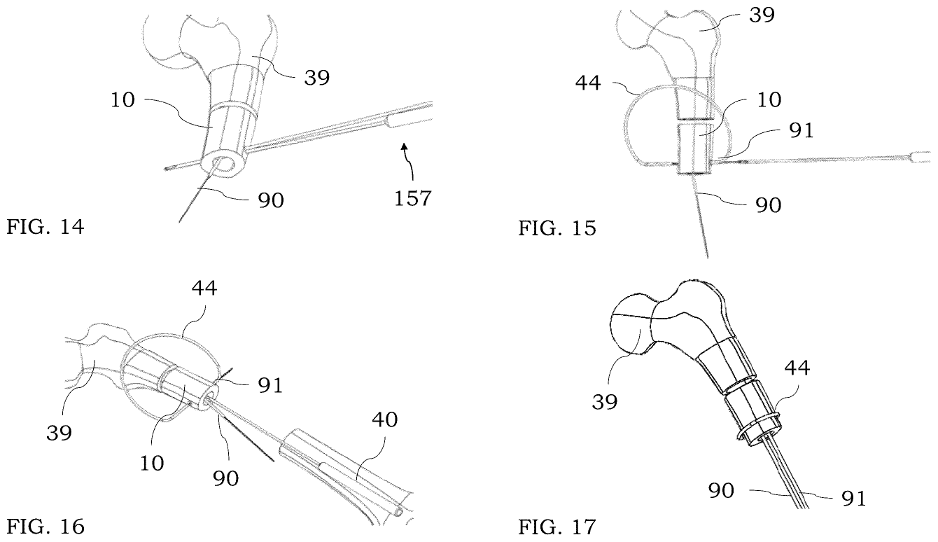

In a step of the surgical method depicted in FIG. 14-17, the second end 91 of the medical cable 9 is also routed through the distal opening of the intramedullary canal 14 via the first tangential bore 11. The routing of the second end 91 may be performed in the same way as the routing of the first end 90, firstly by pulling the loop 92 tangentially through the first tangential bore 11, secondly by pulling it distally through the intramedullary canal 14. At the end of these operations, the medical cable 9 forms the external loop 44 around the transport bone segment 10, while the opposite ends 90, 91 both extend along the anatomical axis, outside of the distal opening of the intramedullary canal 14.

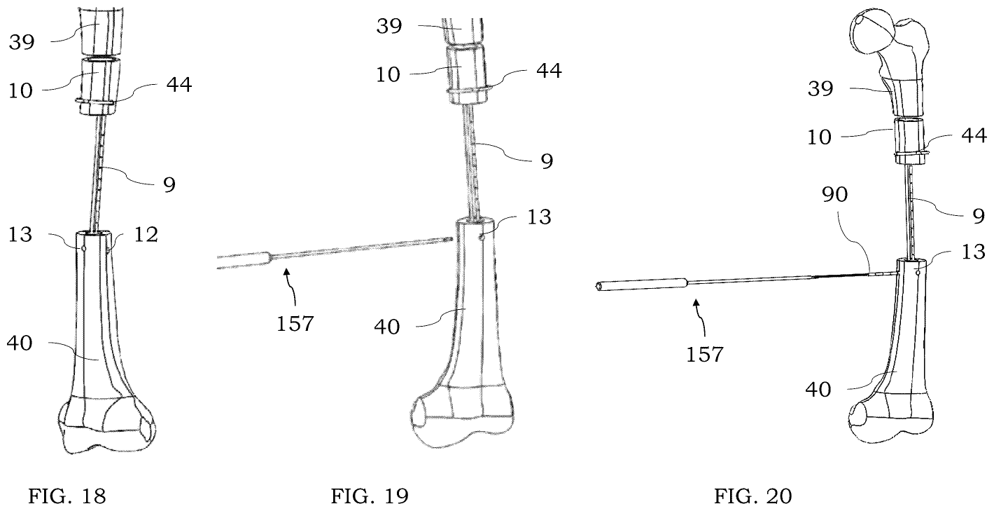

In a step of the surgical method depicted in FIG. 18, a second tangential bore 12 and a third tangential bore 13 are drilled at the fixed distal fixed bone segment 40. Preferably, the second and third tangential bores 12, 13 are drilled along two orthogonal directions, radial to the bone segment. The second tangential bore 12 may be aligned with the first tangential bore 11. The second tangential bore 12 may extend along a medio-lateral direction, while the third tangential bore 13 may extend in an antero-posterior direction. The second tangential bore 12 may be situated slightly more distally, for instance about 3.5 mm more distally, on the distal fixed bone segment 40 with respect to the third tangential bore 13. It is observed that the step of drilling the second and third tangential bores 12, 13 can be performed either before or after the routing of the cable through the first tangential bore 11 and the intramedullary canal 14. Both the second tangential bore 12 and the third tangential bore 13 are preferably bi-cortical bores.

In a step of the surgical method depicted in FIGS. 19-20, the first end 90 and the second end 91 of the medical cable 9 are routed through a proximal opening of the intramedullary canal 14 of the fixed bone segment 40 and finally extracted through the second bores 12. A pulling instrument 157 can once again be employed to engage the loops 92 and pull the ends 90, 91 through the bores. It is observed that the first and second ends 90, 91 should be routed respectively through the opposite second tangential bores 12. The fact that the two opposite ends 90, 91 are visually distinguishable helps the surgeon in discriminating between the two opposite loops 92 when engaging them with the hook 158.

In a final step, not shown in the Figures, the first end 90 and second end 91 of the cable can be fixed to an external fixator structure. At least one end, preferably both, of the medical cable 9 can be wound on the reel of a cable pulling device 8 secured to the external fixator structure.

After extracting the medical cable 9 from the second tangential bores 12a balance screw 43 may be fixed to the distal fixed bone segment 40, in a manner spanning the intramedullary canal, through the third tangential bores 13. The balance screw 43 may act as a fulcrum pin to translate horizontal force on the cable 9 into vertical force to pull the transport bone segment 10 during transport. In this regard the first end 90 of the cable 9 may pass distally of the balance screw 43 and exit the distal bone segment 40 through one of the second tangential bores 12 while the second end 91 of the cable 9 may pass distally of the balance screw 43 and exit the distal bone segment 40 through the other second tangential bore 12.

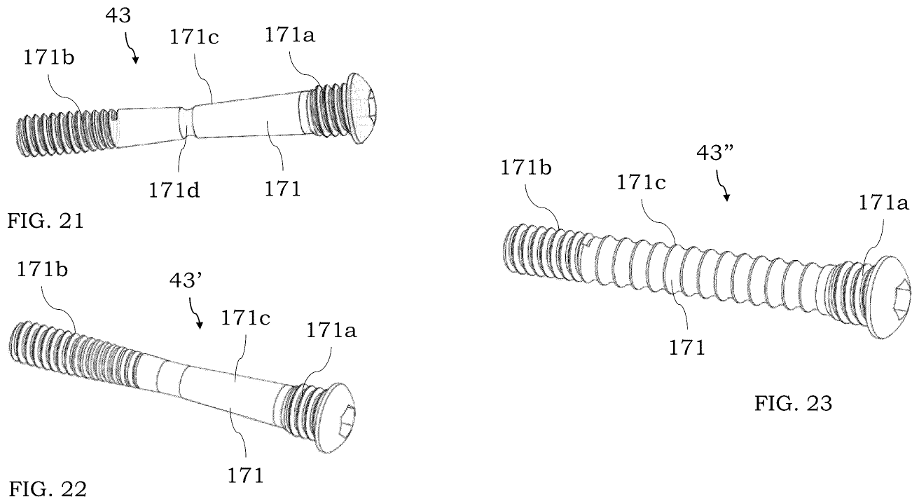

Example of a balance screws 43, 43′, 43″ are shown in FIGS. 21-23. The balance screws 43, 43′, 43″ comprise a shaft 171 with a first threaded region 171a, a second threaded region 171b, and an intermediate region 171c positioned between the first and second threaded regions. The intermediate region 171c may be smooth to reduce friction between the cable 9 and shaft 171. In the example of FIG. 21, the intermediate region 171c includes a guiding groove 171d for guiding the medical cable 9. FIG. 23 illustrates an example of a balance screw 43″ having threads in the intermediate region 171c which have a smaller diameter, and which are dulled as compared to the threads in the first and second threaded regions 171a, 171b. The helical grooves between the threads in the intermediate region 171c may act as cable guides to retain the first end 90 of the medical cable 9 in a first helical groove and the second end 91 of the medical cable 9 in a second helical groove. Balance screws 43, 43′, 43″ may feature a head with a hexagonal recess for a tightening tool.

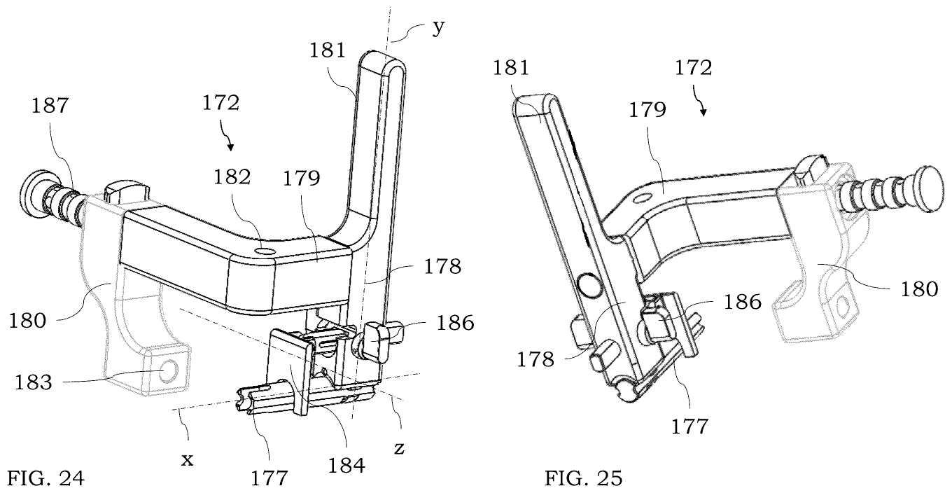

FIGS. 24-27 show a drill guide that may be used in drilling one or more of the first, second, and third tangential bores 11, 12, 13. The drill guide 172 comprises a sleeve 177 integrally attached to a perpendicular first arm 178. The sleeve 177 is configured to be inserted axially into the intramedullary canal 14 of a bone, with the first arm 178 departing tangentially from the bone. Therefore, the sleeve 177 is configured to extend along a first axis x following a proximal-distal direction, and the first arm 178 is configured to extend along a second axis y. A third axis z, orthogonal to the first and second, is also defined. The first arm 178 comprises a handle which extends away from the sleeve 177.

The drill guide 172 further comprises a third arm 180 attached to the second arm and a second arm 179 connecting the first arm 178 to the third arm 180. The second arm is L-shaped with: a first portion, attached to the first arm 178, extending parallel to the first axis x; and a second portion, attached to the third arm 180, perpendicular to the first portion and therefore directed parallel to the third axis z. A first guiding hole 182, parallel to the second axis y and therefore directed towards the sleeve 177, is provided at the junction between the first portion and the second portion. The third arm 180 extends perpendicular to the second portion of the second arm 179, substantially parallel to the first arm 178 i.e. along the second axis y. It bears a second guiding hole 183, directed along the third axis z, at its free end.

A movable abutment 184 is provided which is slidable along the length of the sleeve 177. The movable abutment 184 can be set to adjust the length of the sleeve 177 entering the intramedullary canal 14. The movable abutment 184 is mounted to a guiding rod 185 which slides within a slit of the first arm 178 and which can be locked herein by maneuvering an external key 186.

The distance of the third arm 180 from the first guiding hole 182 can be adjusted along the axis of the second portion of the second arm 179. To this purpose, the third arm 180 is slidingly mounted on a rod 187 integral to said second portion of the second arm 179 and can be locked in a plurality of position along said rod, thanks to a locking mechanism 188 engaging in a plurality of grooves of the rod 187.

In use, the first guiding hole 182 can be used to drill the third tangential hole 13 while the second guiding hole 183 can be used to drill the second tangential hole 12. Also, the sleeve 177 can be used intraoperatively to guide the medical cable 9 while a redirecting pin or balance screw is passed through the first guiding hole 182.

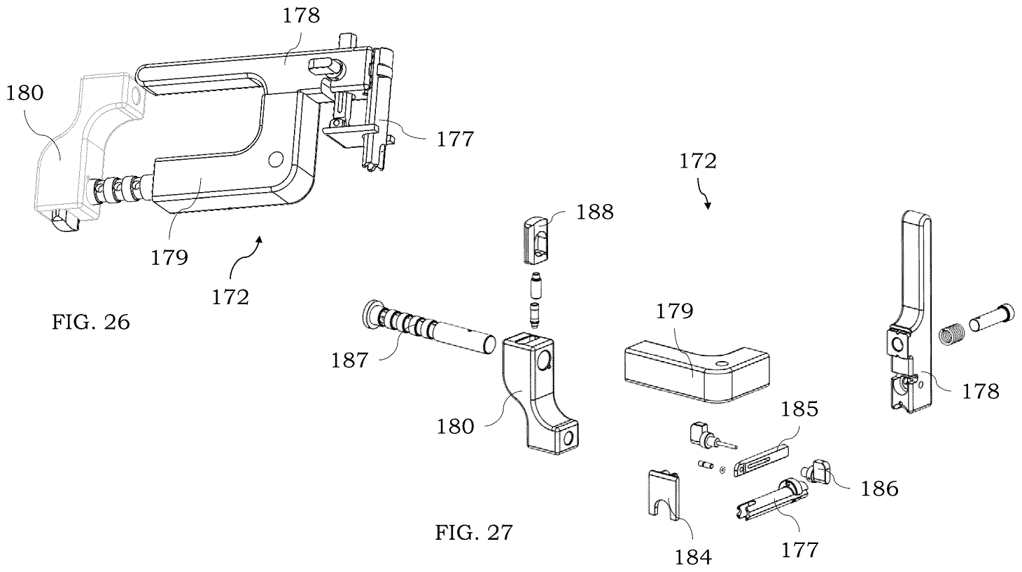

As best seen in FIG. 26, the first arm 178, second arm 179 and third arm 180 can be coupled with rotational joints so that the drill guide can be reconfigured in a planar configuration for ease of packaging.

Although specific examples have been illustrated and described above, those of ordinary skill in the art will appreciate that an arrangement to achieve the same results can be substituted for the specific embodiments shown. This disclosure is intended to cover adaptations or variations of one or more embodiments of the present disclosure. It is to be understood that the above description has been made in an illustrative fashion, and not a restrictive one. The scope of one or more examples of the present disclosure should be determined with reference to the appended claims, along with the full range of equivalents to which such claims are entitled.

What is claimed is:

1. A medical cable, comprising:
a flexible main body having an external cable diameter, the main body having a first end and a second end; and
at least one lead wire extending from the first end and/or from the second end of the main body, the lead wire having a wire diameter and being more flexible than the main body, the wire diameter being less than one half of the cable diameter, said lead wire defining a closed loop.

2. The medical cable according to claim 1, wherein two lead wires defining a closed loop are provided at both the first end and the second end.

3. The medical cable according to claim 2, wherein the first end of the cable is visibly distinguishable from the second end of the cable.

4. The medical cable according to claim 3, wherein the first end and the second end are distinguishable because of a different marking or a difference in color in the main body and/or in the lead wires respectively attached thereto.

5. The medical cable according to claim 1, wherein the wire diameter is less than one third, preferably less than one fifth of the cable diameter.

6. The medical cable according to claim 1, wherein the main body is formed of a plurality of strands, each strand having a plurality of strand wires.

7. The medical cable according to claim 6, wherein the strand wires and the lead wire are made of different materials.

8. The medical cable according to claim 1, wherein said lead wire forming the closed loop extends from the main body for at least 70 mm, preferably for at least 90 mm.

9. The medical cable according to claim 1, further comprising at least a dimensional index at the first end and/or at the second end.

10. An orthopedic bone transport system, comprising:
an external fixation frame adapted to be solidly attached to at least a fixed bone segment;
at least a cable pulling device adapted to be fixed on said external fixation frame;
at least a medical cable adapted to secured to a transport bone segment and to be wound on said cable pulling device, said medical cable having closed loops at both its opposite ends.

11. The orthopedic bone transport system according to claim 10, further comprising a balance screw or pin adapted to be anchored to at least a fixed bone segment to redirect the medical cable within an intramedullary canal of said bone segment.

12. A drill guide comprising:
a sleeve extending along a first axis having a first end portion configured to be received in an intramedullary canal of a bone;
a first arm extending perpendicular to said sleeve along a second axis;
a second arm attached to the first arm at a first joint; the second arm comprising a first guiding bore, the first guiding bore being spaced apart from the first joint along a direction parallel to the first axis;
a third arm attached to the second arm at a second joint, the second joint being spaced apart from the first guiding bore along a direction parallel to a third axis, the third axis being orthogonal both to the first axis and to the second axis; the third arm comprising a second guiding bore, the second guiding bore being spaced apart from the second joint along a direction parallel to the second axis.

13. The drill guide according to claim 12, wherein the first guiding bore is parallel to the second axis and the second guiding bore is parallel to the third axis.

14. The drill guide according to claim 12, wherein the sleeve comprises a movable abutment which can be moved along the first axis to adjust the amount of length of the sleeve insertable in the intramedullary canal.

15. The drill guide according to claim 12, wherein the first joint and the second joint are rotatable so that the first arm, the second arm and the third arm can be set in a planar configuration for storage and shipping.
